(19) United States
(12) Patent Application Publication
(10) Pub. No.: US 2022/0347328 A1
(43) Pub. Date: Nov. 3, 2022

(54) MOBILE DEVICE BASED FAR ULTRA-VIOLET C LED BACTERIA/VIRUS/PATHOGEN ELIMINATOR

(71) Applicant: MICRONAN INC., Anahola, HI (US)

(72) Inventors: William HOZEY, Kailua, HI (US); Kauiokalani KAUHI, Waimanalo, HI (US); Andrew LOCKE, Trabuco Canyon, CA (US)

(21) Appl. No.: 17/614,431

(22) PCT Filed: May 29, 2020
(86) PCT No.: PCT/US2020/035149

§ 371 Date: Nov. 26, 2021

Related U.S. Application Data
(60) Provisional application No. 62/853,943, filed on May 29, 2019.

(51) Int. Cl. A61L 2/10; A61L 9/20

(52) U.S. Cl. CPC A61L 2/10; A61L 9/20; A61L 2202/11; A61L 2209/12; A61L 2209/11; A61L 2202/14; A61L 2202/16

(57) ABSTRACT

A mobile device and method for using a mobile device to disinfect by eliminating bacteria, viruses, and pathogens is provided. The mobile device includes a disinfecting light emitting diode that emits UVC light only in the range of 200 nm to 220 nm to disinfect without harming human tissue. Disinfecting of an object or surface can be accomplished using the mobile device by turning on the LED to provide UVC light in a range of 200-220 nm and holding the mobile device about 0.5 to about 3.0 inches from an object to be disinfected.

### CROSS REFERENCE TO RELATED APPLICATIONS

This application claims the benefit of U.S. Provisional Patent Application No. 62/853,943, filed May 29, 2019, the contents of which is incorporated by reference herein in its entirety.

### FIELD

The present disclosure generally relates to mobile devices and, more particularly, to the use of mobile devices for disinfecting with far UVC light.

### BACKGROUND

Mobile devices, such as cell phones, tablets, and laptop computers have incorporated a multitude of functions into a single device. One function not currently provided by mobile devices is the ability to disinfect using light.

Ultraviolet (UV) light in the UVB range (280-315 nm) and the UVC range (200 nm-280 nm) have been used for disinfecting air, water, and surfaces. Conventional sources of UV light include mercury-vapor lamps and xenon lamps. These lamps, however, emit a very wide range of UV wavelengths, the vast majority of which are not useful for disinfecting and may pose a health hazard. Furthermore, these lamps require a high voltage power supply that may cause safety issues. Conventional light emitting diodes (LEDs) can emit in the UVB and UVC wavelengths. They, however, emit low power, cannot be tuned to the specific wavelengths that are desirable for disinfecting, and are very inefficient and expensive.

It would be desirable to have an efficient, wavelength specific UVC LED incorporated into a mobile device for use in disinfecting.

### SUMMARY

According to the present teachings, a mobile device for disinfecting a surface of an object is provided. The mobile device includes a display, a hardware processor, a disinfecting light emitting diode (LED), and a power supply electrically connected to the display, the hardware processor, and the disinfecting LED. And, the disinfecting LED emits Far UVC light at one or more wavelengths in a range of 200 nm to 220 nm.

The mobile device optionally comprises a power efficiency that ranges from a 1 Watt input yielding about 5 to about 25 milliwatt output. The disinfecting LED of the mobile device can have dimensions ranging from about 4.4 mm×4.4 mm to about 1 mm×1 mm. The disinfecting LED of the mobile device can be disposed on a side of the mobile device opposite the display. The mobile device can further reduce the number of bacteria, viruses, or pathogens on a surface by Log 4.1 or greater after exposure for 5 seconds at 3 cm. The disinfecting LED can be powered by about 0.1 Watts to about 6 Watts. The mobile device can further include at least one electronic processor that executes instructions to perform operations comprising receiving a request generated by a user to disinfect the object; turning on a disinfecting LED disposed in the mobile device, wherein the disinfecting LED provides UVC light at one or more wavelengths within a range of 200-220 nm; causing the mobile device to provide guidance to the user, wherein the guidance comprises a distance from which the object should be from the mobile device; and turning off the disinfecting LED. The disinfecting LED of the mobile device can provide UVC light at 218 nm or 256 nm. The disinfecting LED of the mobile device can comprise a substrate comprising copper and aluminum, a die comprising aluminum-gallium nitrite, and a plurality of silicon quantum dots. And, the mobile device can be one of a cell phone, a tablet, or a laptop computer.

According to the present teachings, provided is a non-transitory computer storage media comprising instructions, when executed by at least one electronic processor of a mobile device, cause the mobile device to perform operations including receiving, at the mobile device, a request generated by a user to disinfect the object; turning on a disinfecting LED disposed in the mobile device, wherein the disinfecting LED provides UVC light at one or more wavelengths within a range of 200-220 nm; providing guidance to the user, wherein the guidance comprises a distance from which the object should be from the mobile device; and turning off the disinfecting LED.

According to the present teachings, the non-transitory computer storage media comprising instructions, when executed by at least one electronic processor of a mobile device, cause the mobile device to optionally open a software application on the mobile device that provides a graphical user interface to control the LED, direct the user to position the mobile device about 0.5 to about 3.0 inches from the object to be disinfected, turn off the disinfecting LED is accomplished without user intervention based on a predetermined time duration, power the disinfecting LED by about 0.1 Watts to about 6 Watts, and turning off the disinfecting LED, and reduce a number of bacteria, viruses, and/or pathogens on the object by Log 4.1 or greater.

According to the present teachings a method of disinfecting using a mobile device is provided. The method includes activating an LED disposed within a mobile device, wherein the LED is configured to output UVC light within a range of 200-220 nm; positioning the mobile device about 0.5 to about 3.0 inches from the object to be disinfected; exposing the object to UVC light within the range of 200-220 nm for about 1 second to about 15 seconds; and turning off the LED.

The method can further include exposing the object to UVC light within the range of 200-220 nm for about 1 second to about 15 seconds to reduce a number of bacteria, viruses, and/or pathogens on the object by Log 4.1 or greater.

It is to be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate the present disclosure and together with the description, serve to explain the principles of the present disclosure.

FIG. 1A schematically depicts a front view of a mobile device according to the present disclosure;

FIG. 1B schematically depicts a rear view of a mobile device according to the present disclosure;

FIG. 2 schematically depicts a rear view of a mobile device with the rear body removed according to the present disclosure;

FIG. 3A schematically depicts a rear view of a mobile device according to the present disclosure;

FIG. 3B schematically depicts a mobile device used to disinfect a surface according to the present disclosure;

FIGS. 4A-B schematically depict a disinfecting LED according to the present disclosure.

FIG. 5 depicts a flow chart of a method for disinfecting according to the present disclosure.

FIG. 6 schematically depicts a mobile device and a software implementation of the method for disinfecting according to the present disclosure.

### DESCRIPTION

Reference will now be made in detail to exemplary implementations of the present disclosure, examples of which are illustrated in the accompanying drawings. Wherever possible, the same reference numbers will be used throughout the drawings to refer to the same or like parts. In the following description, reference is made to the accompanying drawings that form a part thereof, and in which is shown by way of illustration specific exemplary implementations in which the present disclosure may be practiced. These implementations are described in sufficient detail to enable those skilled in the art to practice the present disclosure and it is to be understood that other implementations may be utilized and that changes may be made without departing from the scope of the present disclosure. The following description is, therefore, merely exemplary.

Studies have shown that certain narrow bands of Far UVC light can effectively kill viruses, bacteria, and/or pathogens without damaging the epidermis layer of human skin or the retina. The disclosed mobile device incorporates a disinfecting LED that consumes minimal power and generates minimal heat to provide Far UVC light, for example, at one or more wavelengths in a range from about 200 to about 220 nm to disinfect by killing viruses, bacteria and/or pathogens without damaging human tissue. Because of the small size and inexpensive cost of the disinfecting LED, it can be incorporated into a mobile device.

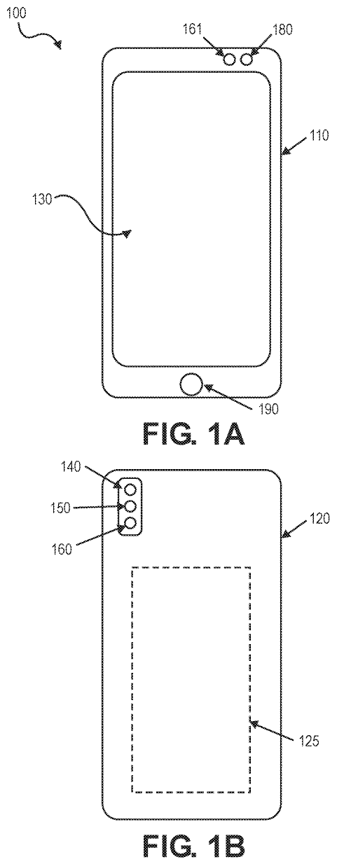

FIGS. 1A-B depict a mobile device 100 according to the present disclosure. Mobile device 100 can be a cellular phone (also referred to herein as a cell phone or mobile phone), a tablet, a laptop computer, or any electronic device that can be carried by a user. FIG. 1A show a front view of mobile device 100 and can include a front body 110 and a display 130.

Front body 110 can be formed of a metal, plastic, glass, ceramic or combinations thereof. Display 130 can be, for example, an LCD, OLED, AMOLED, or microLED display. Mobile device 100 can optionally include a front facing camera 180 and a hardware button 190, both of which are known in the art.

FIG. 1B depicts a back view of mobile device 100 and can include a rear body 120, a rear facing camera 140, a flash LED 150, and a disinfecting LED 160. Mobile device also includes a battery 125. Similar to front body 110, rear body 120 can be formed of a metal, plastic, glass, ceramic or combinations thereof. Rear facing camera 140 and flash LED 150 are known in the art. Front body 110 and rear body 120 can be single unit or formed as a single piece of material in which case front body 110 refers to the front portion of the single unit and rear body 120 refers to the rear portion of the single unit.

Disinfecting LED 160 can provide Far UVC light. For example, disinfecting LED 160 can be tuned to provide light only in a range of 200 nm to 220 nm. Disinfecting LED 160 can optionally provide Far UVC light in a range with the UVC spectrum of 207 nm to 222 nm, or from 210 nm to 215 nm. Disinfecting LED 160 can also be tuned to provide Far UVC light at one or more specific wavelengths within the UVC range of 207 nm to 220 nm, for example, 218 nm, 219 nm, 220 nm, etc. Disinfecting LED 160 can also be tuned to provide UVC light at one or more wavelengths in a range from about 220 to about 280, for example, at 256 nm.

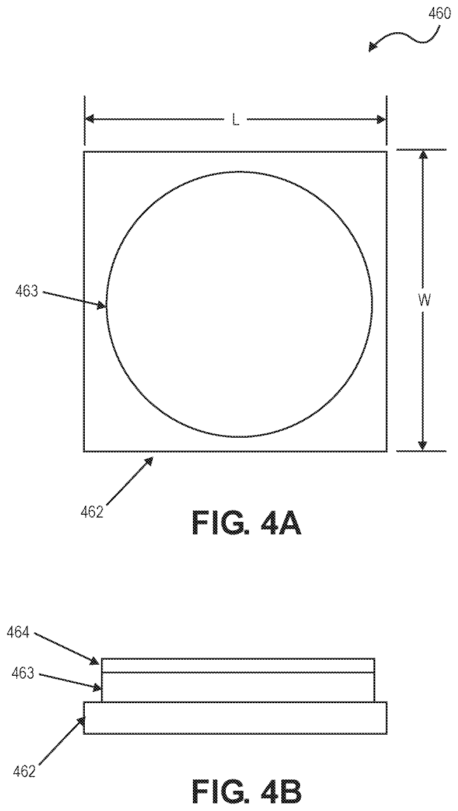

Tuning of disinfecting LED 160 can be accomplished by a combination of a substrate including aluminum gallium nitrate and populated with a plurality of silicon quantum dots. FIG. 4A schematically depicts a top view and FIG. 4B schematically depicts a side view of a disinfecting LED 460 that provides Far UVC light, for example at 218 nm, according to the present disclosure. Disinfecting LED 460 includes a substrate 462, a die 463 disposed on substrate 462, and a plurality of silicon quantum dot filter particles 464 disposed on die 463. In various embodiments, substrate 462 can be formed of a combination of aluminum and copper. Substrate 462 can vary in size from a width W of about 4.40+/−0.15 millimeters and a length L of about 4.40+/−0.15 millimeters to width W of about 1+/−0.05 millimeter and length L of about 1 millimeter+/−0.05. Die 463 can be formed of, for example, aluminum gallium nitrite and disposed on substrate 462. The dimensions of die 463 can vary depending on the size of the substrate, for example, it can have a length and a width that varies from 40 mm×40 mm to less than 1 mm×1 mm and be patterned in either a serial or parallel pattern depending on the UVC wavelength range desired or specific wavelengths desired.

Disinfecting LED 160, also referred to here as a microchip, can have dimensions to fit within mobile device 100, for example 1 mm×1 mm. Power consumption can be about 1 Watt to about 5 Watts. Power efficiency can range from about 1 Watt input yielding about 5 to about 25 milliwatt output, to 1 Watt input yielding about 10 to about 20 milliwatt output, or to 1 Watt input yielding about 15 milliwatt output. The output, at the Far UVC wavelength, can disinfect a surface area of about at least 9.4 cm squared and can increase as the distance from the mobile device to the surface to be disinfected increases. Disinfecting LED 160 can be, for example, an Ultraviolet-C silica quantum dot integrated LED microchip (SQDILM) available from Micronan, Inc. (Honolulu, Hi.).

The present teachings further contemplate a disinfecting LED disposed on a front of mobile device 100 instead of or in addition to disinfecting LED 160 disposed on the back of mobile device 100. FIG. 1A depicts a front view of mobile device 100 including front body 110 and display 130. As in FIG. 1A, front body 110 can be formed of a metal, plastic, glass, ceramic or combinations thereof. Display 130 can be, for example, an LCD, OLED, AMOLED, or microLED display. Mobile device 100 can optionally include front facing camera 161 and a hardware button 190, both of which are known in the art. Mobile device 100 can further include a front facing disinfecting LED 161. Front facing disinfecting LED 161 can be positioned as desired, for example, on front body 110 or within display 130. Front facing disinfecting LED 161 can be similar to disinfecting LED 160 or can optionally provide light at a different wavelengths or wavelength ranges than disinfecting LED 160. For example, front facing disinfecting LED 161 can be tuned to provide from light at one or more wavelengths in a range from about 200 nm to about 220 nm that are the same or different than disinfecting LED 160. Front facing disinfecting LED 161 can also be tuned to provide UVC or Far UVC light at specific wavelengths, for example, 218 nm or 256 nm.

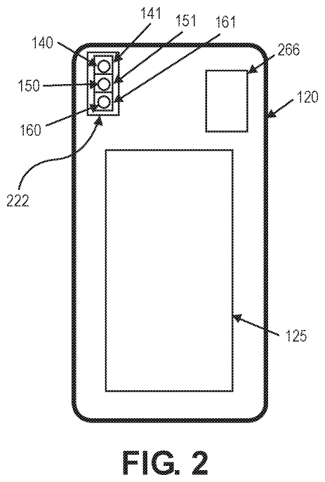

FIG. 2 shows a rear view of mobile device 100 with rear body 120 removed. In an exemplary embodiment, each of disinfecting LED 160, rear facing camera 140, and flash LED 150 can be separate modules. For example, disinfecting LED 160 can be part of disinfecting LED module 161, rear facing camera 140 can be part of rear facing camera module 141, and flash LED 150 can be part of flash LED module 151. Each of disinfecting LED module 161, rear facing camera module 141, and flash LED module 151 can be arranged in a common housing 222. Mobile device can further include a hardware processor 266. Hardware processor 266, for example, executes instructions to perform tasks and processes of mobile device 100.

Power to operate disinfecting LED 160 can be provided, for example, by battery 125. In various embodiments, battery 125 can provide sufficient power for disinfecting LED 160 to operate, for example, at about 0.1 to about 6 watts. Optionally, LED 160 can have a power consumption of about 1 to about 5 watts or from about 2 to about 4 watts. In various embodiments, no capacitor is needed. Heat generated by, for example, a 2 Watt or 5 Watt LED is minimal due to the short periods of time required to disinfect.

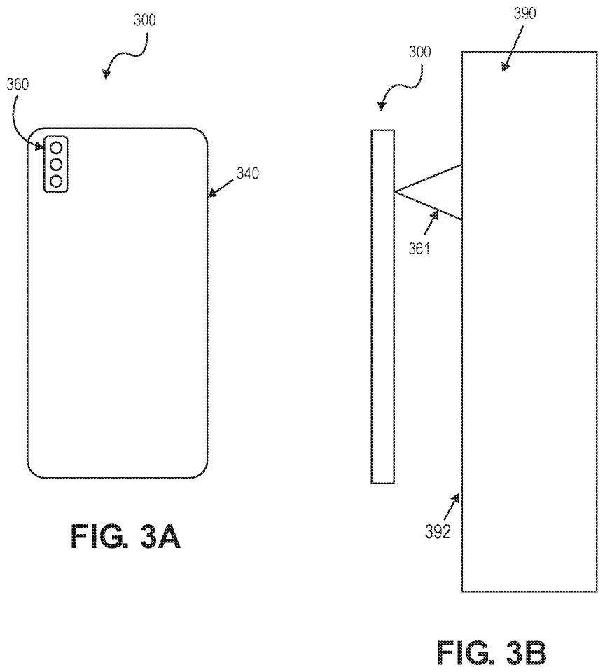

FIGS. 3A-B schematically depict a mobile device 300 disinfecting an object 390, such as, a surface 392 of object 390. As shown in FIG. 3A, the back of mobile device 300 can include a disinfecting LED 360 disposed on a rear body 340 of mobile device 300. FIG. 3B shows a side view of mobile device 300 with a UVC light 361 being emitted from disinfecting LED 360. Exposure of object 390 to UVC light 361 can disinfect surface 392 by reducing the number of bacteria, viruses, and pathogens on surface 392. In an embodiment, disinfecting can be accomplished by positioning mobile device 300 about 0.5 inches to about 3 inches from surface 392. Disinfecting LED 360 can be turned on to expose surface 392 to UVC light 361 for about 1 seconds to about 15 seconds or more.

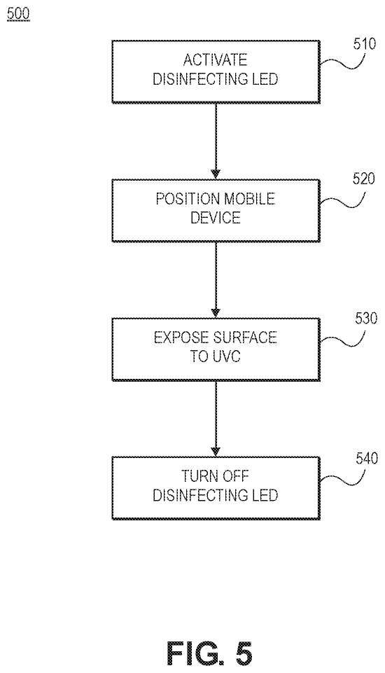

A method for disinfecting 500 according to the present disclosure is depicted in FIG. 5. At 510 of method 500, a disinfecting LED is activated. The disinfecting LED that is activated is disposed on a mobile device, for example, as shown in FIGS. 1A, 1B, 2, 3A and 3B, and is a disinfecting LED, for example, such as disinfecting LED 160 as disclosed herein. Activating the disinfecting LED can be accomplished using a dedicated switch on the mobile device or using a software application. Activating the disinfecting LED can further include selected one or more predetermined exposure times, for example, 5 seconds, 6 seconds, etc.

At 520 of method 500, the mobile device is positioned proximate to a surface of an object to be disinfected. The distance from the surface of the object to be disinfected to the mobile device can be, for example, about 0.5 inches to about 3 inches. One or ordinary skill in the art will understand that positioning the mobile device can also be done at the same time or prior to activating the disinfecting LED at 510 of method 500.

At 530 of method 500, the surface of the object to be disinfected is exposed to UVC light. The UVC light can be Far UVC light having one or more wavelengths in a range of 200 to 220 nm or can be UVC light having one or more wavelengths in a range of 220 nm to 280 nm. Exposure time can range from about 1 second to about 15 seconds.

At 540 of method 500, the disinfecting LED can be turned off. Turning off the disinfecting LED can be accomplished using a dedicated switch on the mobile device or using a software application. For example, the disinfecting LED can be turned off as desired or if a predetermined exposure time was selected at 510 of method 500, the disinfecting LED can be turned off automatically after the predetermined exposure time has elapsed. After turning off disinfecting LED, a number of bacteria, viruses, and/or pathogens on the surface of the object is reduced by Log 4.1 or greater.

### EXAMPLE 1

The effectiveness of the disclosed method and mobile device to eliminate viruses, bacteria and/or pathogens was demonstrated by testing. A first disinfecting LED outputting UV light at 218 nm and a second disinfecting LED outputting UV light at 256 nm were used. Testing was conducted on two bacterial specimens, E. coli and G. Stearothermophilus. The G. Stearothermophilus spore is one of the hardest bacteria to eliminate and is the bacterial standard to ensure medical sterilization of medical instruments. For each test, the bacterial specimens were placed in two petri dishes, a first for testing and a second for use as a control. The number of colony forming units (CFUs) present in the petri dishes was then counted for each test and a control specimen.

The Log is a measurement of how many CFU/Bacteria there are; as the log increases, the number of CFU/Bacterial becomes multiplied by 10. For example, Log 1 has 10 CFU's versus Log 2 which has 100 CFU's, versus Log 5 which has 100,000 CFU's. As shown in Table 2, the Log Reductions convey how effective a product is at reducing pathogens. The greater the log reduction the more effective the product is at killing bacteria and other pathogens that can cause infections. For Reference, Log 6 reduction is considered medical sterility because it kills 99.9999% of bacteria.

TABLE 2

| | | Percentage | |
|---|---|---|---|
| Log reduction | Number of CFUs | reduction | Times smaller |
| 0 log (Log0) | 1000000 | 0% | N/A |
| 1 log (Log1) | 100000 | 90% | x 10 |
| 2 log (Log2) | 10000 | 99% | x 100 |
| 3 log (Log3) | 1000 | 99.9% | x 1000 |
| 4 log (Log4) | 100 | 99.99% | x 10000 |
| 5 log (Log5) | 10 | 99.999% | x 100000 |
| 6 log (Log6) | 1 | 99.9999% | x 1000000 |

The disinfecting LEDs were positioned 1 cm above the bacteria for all the tests. A lens was used to direct the UV light from the disinfecting LEDs to the bacterial specimens. For both disinfecting LEDs, the wattage and the exposure times were varied. The results are shown below in Table 2.

TABLE 2

| LED | Exposure | Microorganism | Log Reduction |
|---|---|---|---|
| 256 nm, 5 W | 15 seconds | Geobacillus | 3.2 |
| | | stearothermophilus | |
| 218 nm, 5 W | 10 seconds | E. coli | 3.2 |
| 218 nm, 3 W | 10 seconds | E. coli | 3.0 |
| 218 nm, 1 W | 10 seconds | E. coli | 2.9 |
| 256 nm, 3 W | 10 seconds | E. coli | 3.5 |

After exposure to the UV light, the number of CFUs present in the test and control specimens was counted. The difference between the number of CFUs present in the control specimen and the number of CFUs present in the test specimen was then expressed as a log reduction. As shown in Table 1, a log reduction of 2 correlates to a 99% reduction of CFUs, a log reduction of 3 corrections to a 99.9% reduction of CFUs, and a 4 log reduction correlates to a 99.99% reduction of CFUs. As shown in Table 2, all exposure times and wattages, for both the 218 nm and 256 nm disinfecting LEDs, effectively eliminated 99% to 99.99% of the bacterial specimens.

### EXAMPLE 2

Testing was performed using an Ultraviolet-C silica quantum dot integrated LED microchip (SQDILM) available from Micronan (Honolulu, Hi.) at 218 nm wavelength. Four separate test were conducted, including two different wattage tests at similar distances, one different wattage test at different time and distance, and a positive control to be used to obtain a known response, so that this positive response could be compared to the unknown response of the experiment.

All the tests, including the positive control test, were performed on the common infective bacteria, E. Coli. The bacteria in all tests were placed on approximately 10 mm by 10 mm square slides, allowed to dry, then were either exposed with the UV light (3 slides) or allowed to grow naturally for the positive control (1 slide) test.

CFUs (Coupon or Colony Forming Units), representing the number of Bacterial Colonies formed in each sample slide including the control slides, were determined after the experiment was concluded and reported in terms of Log reduction as shown in Table 1.

An untreated bacterial slide, used as a Positive Control, had a large number of CFU's as seen below.

| Microorganism | CFU/Coupon | Log |
|---|---|---|
| E. Coli | 1.6 × 106 | 6.1 |

This showed that there are 1,600,000 bacterial colonies formed from the E. Coli.

During testing the Positive Control was compared to the 218 nm Wavelength UV-C emitting LED light at different distances, different times and to different LED Wattage.

Test 1- 218 nm at 1.25 Watts on E. coli
at 3 cm distance for 5 Seconds

| | Distance | CFU/Coupon | | Log |
|---|---|---|---|---|
| Exposed Time | from slide | (slide) | Log | Reduction |
| 5 seconds | 3 centimeters | <100 | <2 | ≥4.1 |

This showed that in comparing this test to the positive control, the E. Coli went from having a log 6.1 in the positive control to a log of less than 2.0, an equal to or greater than 4.1 Log reduction. In essence, the amount of E. coli was 10000 times smaller or eliminated over 99.99% of all the bacteria in this slide experiment in 5 seconds at 3 centimeters away from our LED UV-C source using only 1.25 Watts to power the light.

Test 2- 218 nm at 1.5 Watts on E. Coli
at 3 cm distance for 5 Seconds

| | Distance | CFU/Coupon | | Log |
|---|---|---|---|---|
| Exposed Time | from slide | (slide) | Log | Reduction |
| 5 seconds | 3 centimeters | <100 | <2 | ≥4.1 |

This showed that in comparing this test to the positive control, the E. Coli went from having a log 6.1 in the positive control to a log of less than 2.0, an equal to or greater than 4.1 Log reduction. In essence, the amount of number of E. Coli was 10000 times smaller or eliminated over 99.99% of all the bacteria in this slide experiment in 5 seconds at 3 centimeters away from our LED UV-C source using only 1.5 Watts to power the light.

Test 3- 218 nm at 1.5 Watts on E. Coli
at 3 inches distance for 7 Seconds

| | Distance | CFU/Coupon | | Log |
|---|---|---|---|---|
| Exposed Time | from slide | (slide) | Log | Reduction |
| 7 seconds | 3 inches | 200 | 2.3 | 3.8 |
| | (7.62 cm) | | | |

Comparing this test to the positive, the E. Coli went from having a log 6.1 in the positive control to a log of 2.3, a 3.8 Log reduction. In essence, the amount of E. Coli was over 1000 times smaller or eliminated well over 99.9% of all the bacteria in this slide experiment in 7 seconds at 3 inches away from our LED UV-C source using only 1.5 Watts to power the light.

In various embodiments, mobile device 100 can further include a software or hardware application to allow a user to control disinfecting LED 160 and to implement method 500. The software application can be, for example, a non-transitory computer readable medium storing instructions, that when executed by a hardware processor, performs a method of providing a graphical user interface on the display to allow a user to control disinfecting LED 160. For example, the software application can be similar to a flashlight app that allows a user to turn on and off the flash LED. A disinfecting app can allow the user to turn on and off disinfecting LED 160 as well as provide instructions relating to distance and time of exposure for disinfecting. Optionally, the software application can automatically turn on the disinfecting LED and turn off the disinfecting LED after the predetermined exposure time.

The software application can, for example, include a non-transitory computer storage medium storing instructions configured to instruct the mobile device to perform 510 of method 500 upon receiving, at the mobile device, a request generated by a user to disinfect the object. Instructions can then be provided to turn on the disinfecting LED disposed in the mobile device, wherein the disinfecting LED provides UVC light at one or more wavelengths within a range of 200-220 nm. For example, the software application on the mobile device can provide a graphical user interface to control the LED that includes an ON/OFF switch and/or buttons to select the predetermined exposure times.

The software application can then provide instructions to implement 520 and 530 of method 500 by providing guidance to the user, wherein the guidance comprises a distance from which the object should be from the mobile device. And, instructions to turn off disinfecting LED at 540 of method 500 can be implanted in software based on expiration of the predetermined exposure time or by providing guidance to the user to that the exposure time should be sufficient for disinfecting the surface of the object.

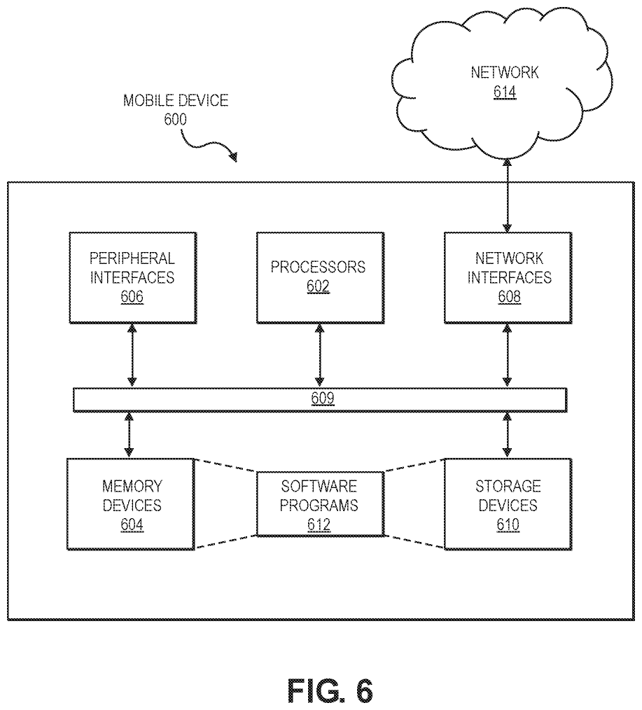

FIG. 6 is an example of a hardware configuration for a mobile device 600, which can be used to perform one or more of the processes described above. Mobile device 600 can be any type of mobile devices, such as smart telephones, laptop computers, tablet computers, cellular telephones, personal digital assistants, etc. As illustrated in FIG. 6, mobile device 600 can include one or more processors 602 of varying core configurations and clock frequencies. Mobile device 600 can also include one or more memory devices 604 that serve as a main memory during the operation of the mobile device 600. For example, during operation, a copy of the software that supports the above-described operations can be stored in the one or more memory devices 604. The mobile device 600 can also include one or more peripheral interfaces 606, such as keyboards, mice, touchpads, computer screens, touchscreens, etc., for enabling human interaction with and manipulation of mobile device 600.

Mobile device 600 can also include a data bus 609, one or more storage devices 610 of varying physical dimensions and storage capacities, such as flash drives, hard drives, random access memory, etc., for storing data, such as images, files, and program instructions for execution by the one or more processors 602. Mobile device 600 can also include one or more network interfaces 608 for communicating via one or more networks, such as Ethernet adapters, wireless transceivers, or serial network components, for communicating over wired or wireless media using protocols.

Additionally, mobile device 600 can include one or more software programs 612 that enable the functionality described above. The one or more software programs 612 can include instructions that cause the one or more processors 602 to perform the processes, functions, and operations described herein. Copies of the one or more software programs 612 can be stored in the one or more memory devices 604 and/or on in the one or more storage devices 610. Likewise, the data utilized by one or more software programs 612 can be stored in the one or more memory devices 604 and/or on in the one or more storage devices 610.

If implemented in software, the functions can be stored on or transmitted over a computer-readable medium as one or more instructions or code. Computer-readable media includes both tangible, non-transitory computer storage media and communication media including any medium that facilitates transfer of a computer program from one place to another. A storage media can be any available tangible, non-transitory media that can be accessed by a computer. By way of example, and not limitation, such tangible, non-transitory computer-readable media can comprise RAM, ROM, flash memory, or EEPROM. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Combinations of the above should also be included within the scope of computer-readable media.

In one or more exemplary embodiments, the functions described can be implemented in hardware, software, firmware, or any combination thereof. For a software implementation, the techniques described herein can be implemented with modules (e.g., procedures, functions, subprograms, programs, routines, subroutines, modules, software packages, classes, and so on) that perform the functions described herein. A module can be coupled to another module or a hardware circuit by passing and/or receiving information, data, arguments, parameters, or memory contents. Information, arguments, parameters, data, or the like can be passed, forwarded, or transmitted using any suitable means including memory sharing, message passing, token passing, network transmission, and the like. The software codes can be stored in memory units and executed by processors. The memory unit can be implemented within the processor or external to the processor, in which case it can be communicatively coupled to the processor via various means as is known in the art.

In one or more exemplary embodiments, the functions described can be implemented in hardware, software, firmware, or any combination thereof. For a software implementation, the techniques described herein can be implemented with modules (e.g., procedures, functions, subprograms, programs, routines, subroutines, modules, software packages, classes, and so on) that perform the functions described herein. A module can be coupled to another module or a hardware circuit by passing and/or receiving information, data, arguments, parameters, or memory contents. Information, arguments, parameters, data, or the like can be passed, forwarded, or transmitted using any suitable means including memory sharing, message passing, token passing, network transmission, and the like. The software codes can be stored in memory units and executed by processors. The memory unit can be implemented within the processor or external to the processor, in which case it can be communicatively coupled to the processor via various means as is known in the art.

Mobile device 600 can communicate with other devices via a network 614. The other devices can be any types of devices as described above. The network 614 can be any type of network, such as a local area network, a wide-area network, a virtual private network, the Internet, an intranet, an extranet, a public switched telephone network, an infrared network, a wireless network, and any combination thereof. The network 614 can support communications using any of a variety of commercially-available protocols, such as TCP/IP, UDP, OSI, FTP, UPnP, NFS, CIFS, AppleTalk, and the like. The network 614 can be, for example, a local area network, a wide-area network, a virtual private network, the Internet, an intranet, an extranet, a public switched telephone network, an infrared network, a wireless network, and any combination thereof.

While the present teachings have been illustrated with respect to one or more implementations, alterations and/or modifications can be made to the illustrated examples without departing from the spirit and scope of the appended claims. For example, it will be appreciated that while the process is described as a series of acts or events, the present teachings are not limited by the ordering of such acts or events. Some acts may occur in different orders and/or concurrently with other acts or events apart from those described herein. For example, steps of the methods have been described as first, second, third, etc. As used herein, these terms refer only to relative order with respect to each other, e.g., first occurs before second. Also, not all process stages may be required to implement a methodology in accordance with one or more aspects or implementations of the present teachings. It will be appreciated that structural components and/or processing stages can be added or existing structural components and/or processing stages can be removed or modified. Further, one or more of the acts depicted herein may be carried out in one or more separate acts and/or phases. Furthermore, to the extent that the terms “including,” “includes,” “having,” “has,” “with,” or variants thereof are used in either the detailed description and the claims, such terms are intended to be inclusive in a manner similar to the term “comprising.” The term “at least one of” is used to mean one or more of the listed items can be selected. As used herein, the term “one or more of” with respect to a listing of items such as, for example, A and B, means A alone, B alone, or A and B. The term “at least one of” is used to mean one or more of the listed items can be selected. Further, in the discussion and claims herein, the term “on” used with respect to two materials, one “on” the other, means at least some contact between the materials, while “over” means the materials are in proximity, but possibly with one or more additional intervening materials such that contact is possible but not required. Neither “on” nor “over” implies any directionality as used herein. The term “about” indicates that the value listed may be somewhat altered, as long as the alteration does not result in nonconformance of the process or structure to the illustrated implementation. Finally, “exemplary” indicates the description is used as an example, rather than implying that it is an ideal. Other implementations of the present teachings will be apparent to those skilled in the art from consideration of the specification and practice of the disclosure herein. It is intended that the specification and examples be considered as exemplary only, with a true scope and spirit of the present teachings being indicated by the following claims.

What is claimed is:

1. A mobile device comprising:
a display;
a hardware processor;
a disinfecting light emitting diode (LED); and
a power supply electrically connected to the display, the hardware processor, and the disinfecting LED,
wherein the disinfecting LED emits UVC light at one or more wavelengths in a range of 200 nm to 220 nm.

2. The mobile device of claim 1, wherein the disinfecting LED comprises a power efficiency that ranges from a 1 Watt input yielding about 5 to about 25 milliwatt output.

3. The mobile device of claim 1, wherein the disinfecting LED has dimensions ranging from about 4.4 mm×4.4 mm to about 1 mm×1 mm.

4. The mobile device of claim 1, wherein the disinfecting LED is disposed on a side of the mobile device opposite the display.

5. The mobile device of claim 1, wherein the disinfecting LED reduces a number of bacteria, viruses, or pathogens on a surface by Log 4.1 or greater after exposure for 5 seconds at 3 cm.

6. The mobile device of claim 5, wherein the disinfecting LED is powered by about 0.1 Watts to about 6 Watts.

7. The mobile device of claim 5, wherein the disinfecting LED is powered by about 1.25 Watts.

8. The mobile device of claim 1, further comprising at least one electronic processor that executes instructions to perform operations comprising:
receiving a request generated by a user to disinfect the object;
turning on a disinfecting LED disposed in the mobile device, wherein the disinfecting LED provides UVC light at one or more wavelengths within a range of 200-220 nm;
causing the mobile device to provide guidance to the user, wherein the guidance comprises a distance from which the object should be from the mobile device; and
turning off the disinfecting LED.

9. The mobile device of claim 1, wherein the disinfecting LED provides UVC light at 218 nm or 256 nm.

10. The mobile device of claim 1, wherein the disinfecting LED comprises a substrate comprising copper and aluminum, a die comprising aluminum-gallium nitrite, and a plurality of silicon quantum dots.

11. The mobile device of claim 1, wherein the mobile device is one of a cell phone, a tablet, or a laptop computer.

12. A non-transitory computer storage media comprising instructions, when executed at least one electronic processor of a mobile device, cause the mobile device to perform operations comprising:
receiving, at the mobile device, a request generated by a user to disinfect the object;
turning on a disinfecting LED disposed in the mobile device, wherein the disinfecting LED provides UVC light at one or more wavelengths within a range of 200-220 nm;
providing guidance to the user, wherein the guidance comprises a distance from which the object should be from the mobile device; and
turning off the disinfecting LED.

13. The computer readable media of claim 12, wherein the mobile device is one of a cell phone, tablet, or laptop computer.

14. The computer readable media of claim 12, wherein turning on the disinfecting LED comprises opening a software application on the mobile device that provides a graphical user interface to control the LED.

15. The computer readable media of claim 12, wherein providing guidance comprises directing the user to position the mobile device about 0.5 to about 3.0 inches from the object to be disinfected.

16. The computer readable media of claim 12, wherein turning off the disinfecting LED is accomplished without user intervention based on a predetermined time duration.

17. The computer readable media of claim 12, wherein turning on a disinfecting LED disposed in the mobile device comprises powering the disinfecting LED by about 0.1 Watts to about 6 Watts.

18. The computer readable media of claim 12, wherein, subsequent to turning off the disinfecting LED, a number of bacteria, viruses, and/or pathogens on the object is reduced by Log 4.1 or greater.

19. A method of disinfecting comprising:
activating an LED disposed within a mobile device, wherein the LED is configured to output UVC light within a range of 200-220 nm;
positioning the mobile device about 0.5 to about 3.0 inches from an object to be disinfected;
exposing the object to UVC light within the range of 200-220 nm for about 1 second to about 15 seconds; and
turning off the LED.

20. The method of claim 19, wherein exposing the object to UVC light within the range of 200-220 nm for about 1 second to about 15 seconds reduces a number of bacteria, viruses, and/or pathogens on the object by Log 4.1 or greater.
